(19) United States
(12) Patent Application Publication
(10) Pub. No.: US 2012/0329813 A1
(43) Pub. Date: Dec. 27, 2012

(54) Pemirolast for the Treatment of Systemic Low Grade Inflammation

(75) Inventors: Johan Raud, Stockholm (SE); Carl-Johan Dalsgaard, Stockholm (SE)

(73) Assignee: Cardoz AB, Stockholm (SE)

(21) Appl. No.: 13/509,430

(22) PCT Filed: Nov. 12, 2010
(86) PCT No.: PCT/GB2010/002096

§ 371 Date: Aug. 14, 2012

Related U.S. Application Data
(60) Provisional application No. 61/260,949, filed on Nov. 13, 2009.

(51) Int. Cl. A61K 31/519; A61P 29/00

(52) U.S. Cl. 514/259.41

(57) ABSTRACT

According to the invention there is provided pemirolast, or a pharmaceutically acceptable salt thereof, for use in the treatment of systemic low-grade inflammation.

### FIELD OF THE INVENTION

This invention relates to a new use of a known mast cell inhibiting compound.

### BACKGROUND AND PRIOR ART

Inflammation is typically characterised as a localised tissue response to e.g. invasion of microorganisms, certain antigens, damaged cells or physical and/or chemical factors. The inflammatory response is normally a protective mechanism which serves to destroy, dilute or sequester both the injurious agent and the injured tissue, as well as to initiate tissue healing.

Many conditions/disorders are characterised by, and/or caused by, abnormal, tissue-damaging inflammation. Such conditions are typically characterized by activation of immune defence mechanisms, resulting in an effect that is more harmful than beneficial to the host, and are generally associated with varying degrees of tissue redness or hyperemia, swelling, hyperthermia, pain, itching, cell death, tissue destruction, cell proliferation and/or loss of function. Examples include inflammatory bowel diseases, rheumatoid arthritis, multiple sclerosis, psoriasis, glomerulonephritis and transplant rejection.

Typically, a complex series of events results in inflammatory changes such as increased blood flow through dilation of local blood vessels, resulting in redness and heat, the extravasation of leukocytes and plasma, often resulting in localised swelling, activation of sensory nerves (resulting in pain in some tissues) and loss of function. These inflammatory changes are triggered by a cascade of cellular and biochemical events involving cells like neutrophils, monocytes, macrophages and lymphocytes together with inflammatory mediators such as vasoactive amines, cytokines, complement factors and reactive oxygen species.

Most inflammatory reactions remain local without causing systemic effects such as fever and chills. However, in some situations, inflammation is widespread or intensive so that inflammatory mediators increase in the circulating blood and begin to affect the entire body. An example of this is bacterial pneumonia which is generally associated with systemic symptoms like high fever (when inflammatory mediators/stimuli reach the brain), chills and/or malaise.

Such a reaction is typically termed “systemic” inflammation. In such situations, proinflammatory cytokines (mainly from monocytes/macrophages; see e.g. Eklund, Adv. Clin. Chem., 48, 111 (2009)) also reach the liver, which responds by producing so-called acute-phase reactants that are released into the blood. The best known of the acute-phase proteins that are released is C-reactive protein (CRP). This, along with other acute-phase reactants, may help to limit tissue injury, to enhance host resistance to infection, and to promote tissue repair and resolution of inflammation (see The Merck Manual of Diagnosis and Therapy, 18^{th} edition (2006)).

The plasma CRP level is a useful marker of inflammation and is routinely measured in both medical and veterinary clinical practice. The highest plasma levels of CRP (often over 100 mg/L) are typically seen in severe bacterial infections. Exacerbations of inflammatory and/or autoimmune diseases like inflammatory bowel disease and rheumatoid arthritis are also associated with high CRP levels (often around 50 mg/L). Likewise, CRP levels in cancer patients are sometimes markedly increased, while mild inflammation and many viral infections cause plasma CRP concentrations in the range of 10-50 mg/L.

Using highly sensitive assays to measure low levels of CRP (termed hsCRP), it has been found that the median CRP (or hsCRP) concentration in apparently healthy subjects is in the range 0.6-0.8 mg/L (see Wilkins et al, Clin. Chem., 44, 1358 (1998)). However, hsCRP concentrations in apparently healthy subjects have a skewed distribution. This was illustrated by Shine et al (Clinica Chimica Acta, 117, 13 (1981)), who reported that, among almost 500 sera from normal adult volunteer blood donors, the median hsCRP value was 0.8 mg/L, with a tail of higher values with the 90th percentile at 3 mg/L and the 99th percentile at 10 mg/L.

Given that CRP production is triggered by inflammation, it is considered that these subjects have so-called “systemic low-grade inflammation” (SLGI). Although the exact molecular mechanisms behind SLGI are not yet fully understood, it is considered to be a distinct condition in itself. SLGI can be diagnosed by detecting minor elevations of CRP (CRP between 0.9 and 10 mg/L; see e.g. Ridker et al, N. Engl. J. Med., 352, 20 (2005) and Eklund, Adv. Clin. Chem., 48, 111 (2009)).

It has been shown that the CRP level (in the low concentration range corresponding to SLGI) is a predictor of cardiovascular events (e.g. myocardial infarction and stroke) and indeed is a better predictor of such events than low density lipoprotein (LDL) cholesterol levels (Ridker et al, N. Engl. J. Med., 347, 1557 (2002)), Recently, the JUPITER study was reported. This was a huge randomised, double-blind, placebo-controlled, multicentre trial conducted at 1315 sites in 26 countries. The trial was conducted over 1.9 years. This trial showed that pharmacological reduction of elevated CRP (in the low concentration range corresponding to SLGI) with rosuvastatin (in apparently healthy subjects with normal or low LDL cholesterol) significantly reduces cardiovascular morbidity/mortality (Ridker et al, ibid., 359, 2195 (2008)).

However, statins suffer from the disadvantage that they are not equally effective in all patients and are known to have certain side effects (e.g. changes in liver function, myopathy and rhabdomyolysis). Furthermore, cardiovascular diseases, such as atherosclerosis, remain a major cause of death and disability. Indeed, a recent review article (Briel et al, JAMA, 295, 2046 (2006)) suggests that statins do not reduce serious cardiovascular events during the first four months of treatment in patients with acute coronary syndromes. There is thus a real unmet clinical need for safer and/or more effective treatments of cardiovascular diseases, and in particular for reducing the risk of cardiovascular morbidity and/or mortality.

SLGI may also predict, and be involved in the development of type 2 diabetes mellitus in initially healthy subjects (see Pickup, Diabetes Technol. Ther., 8, 1 (2006)).

As mentioned above, the etiology of SLGI is still unclear. There is certainly no known direct link between mast cell activity and SLGI/hsCRP levels. In fact, a lack of correlation between circulating levels of mast cell tryptase and CRP has been found in patients with cardiovascular disease (see van Haelst et al, Int. J. Cardiol., 78, 75 (2001) and Kervinen et al, ibid. 104, 138 (2005)), supporting the concept that SLGI is unrelated to mast cell activation. (Tryptase is abundant in mast cell secretory granules and plasma tryptase is used as a selective and reliable marker for mast cell activity (see e.g. Payne and Kam, Anaesthesia, 59, 695 (2004)).)

Furthermore, the well known mast cell inhibiting drug ketotifen has been shown not to reduce CRP in subjects with prediabetes and signs of systemic inflammation (elevated serum TNF-alpha, as measured in over half the patients; see Böhmer et al, Diabetes Care, 17, 139 (1994)). More recently, theophylline, a phosphodiesterase inhibitor known to inhibit mast cell activation has been shown not to reduce hsCRP in patients with chronic obstructive pulmonary disease (Kanehara et al, Pulmonary Pharmacology & Therapeutics, 21, 874 (2008)). From the literature therefore, there is no basis to expect an anti-allergic and/or anti-asthmatic drug that inhibits mast cells to have any effect on SLGI.

It is surprising therefore that we have found that the anti-allergic/anti-asthmatic mast cell-inhibiting drug pemirolast markedly reduces CRP levels subjects with plasma CRP >0.9 mg/L. Such reductions have been observed in apparently healthy non-allergic/non-asthmatic subjects, as well as in subjects with pre-existing cardiovascular conditions. It is therefore considered that pemirolast may be useful in the treatment of SLGI characterised by CRP levels above which the risk for cardiovascular events (e.g. morbidity and/or mortality) has been shown to be increased (see Ridker et al, N. Engl. J. Med., 352, 20 (2005)).

### DISCLOSURE OF THE INVENTION

According to a first aspect of the invention there is provided pemirolast, or a pharmaceutically acceptable salt thereof, for use in the treatment of SLGI.

The term “SLGI” will be understood to include those conditions referred to in the literature variously as “systemic low-grade inflammation”, “low-grade systemic inflammation”, “subclinical systemic inflammation”, “chronic low-grade inflammation”, “persistent low-grade inflammation” or, depending upon the context, just “low-grade inflammation” or “systemic inflammation” (see, for example März et al, Circulation, 110, 3068 (2004) and Nicklas et al, CMAJ, 172, 1199 (2005)). Although other inflammatory markers (e.g. circulating cytokines, adhesion molecules and white blood cells) are known to be indicative of SLGI and may be measured, and reduced, in accordance with the invention, SLGI is always characterised by inter alia plasma CRP levels in subjects (and for example in otherwise outwardly healthy and/or non-allergic/non-asthmatic mammalian subjects) which are less than about 10 mg/L, but which levels are above about 7 mg/L, for example above about 5 mg/L, preferably above about 3 mg/L, more preferably above about 2 mg/L, particularly above about 1 mg/L and more particularly above about 0.9 mg/L. Such plasma CRP levels may be reduced by administration of an appropriate pharmacologically-effective amount of pemirolast or a pharmaceutically acceptable salt thereof.

According to a second aspect of the invention there is provided a method of treatment of SLGI, which method comprises the administration of a pharmacologically-effective amount of pemirolast, or a pharmaceutically acceptable salt thereof, to a patient in need of such treatment.

For the avoidance of doubt, in the context of the present invention, the terms “treatment”, “therapy” and “therapy method” include the therapeutic, or palliative, treatment of patients in need of, as well as the prophylactic treatment and/or diagnosis of patients which are susceptible to, SLGI, or other relevant conditions mentioned herein.

“Patients” include mammalian (including human) patients.

According to two further aspects of the invention there is provided pemirolast, or a pharmaceutically acceptable salt thereof, for the reduction of plasma CRP levels in a patient (to below any one of the values mentioned hereinbefore), as well as a method of reduction of plasma CRP levels in a patient (to below any one of the values mentioned hereinbefore), which comprises administering pemirolast, or a pharmaceutically acceptable salt thereof, to a patient.

As mentioned hereinbefore, SLGI is known to be linked to, for example, metabolic syndrome, diabetes mellitus (e.g. type 2 diabetes), insulin resistance syndrome, obesity, cardiovascular diseases (e.g. atherosclerosis, abdominal aortic aneurysms and other cardiovascular events), and some cancers (e.g. colon cancer). Minor elevation in CRP levels may also be the only sign of disease in otherwise apparently healthy subjects.

Minor elevations in CRP can also predict undesired outcomes or complications (e.g. events) in various medical conditions, or likelihood of dying in different diseases. In particular elevations in CRP may predict events such as cardiovascular morbidity and mortality, and/or the development of type 2 diabetes mellitus, the risk of both of which may, in accordance with the invention, be reduced with pemirolast, or a pharmaceutically acceptable salt thereof.

According to a further aspect of the invention there is provided a method of reducing the risk of (i.e. preventing) cardiovascular morbidity and mortality, and/or of reducing (i.e. preventing) the development of type 2 diabetes mellitus, in a patient, which method comprises:

(a) measuring a plasma CRP level in that patient;

(b) determining whether the level of plasma CRP is above one of the values mentioned hereinbefore, and particularly above about 0.9 mg/L; and

(c) if so, administering pemirolast, or a pharmaceutically acceptable salt thereof, to that patient for a time and at an appropriate dosage to reduce the CRP level, for example to below the relevant value mentioned hereinbefore.

The American Heart Association (AHA) and the Centers for Disease Control and Prevention (CDC) have evaluated CRP as a risk assessment tool and suggested that cut points of below 1 mg/L, between 1 and 3 mg/L, and greater than 3 mg/L be used to identify subjects at lower, average and high relative risk, of developing cardiovascular morbidity or mortality, respectively.

The term “morbidity” will be understood by the skilled person to include any diseased state, disability, illness and/or poor health generally. “Cardiovascular” morbidity therefore includes such states exhibited as a consequence of an underlying cardiovascular complication, which may in itself be a consequence of one or more of the other conditions mentioned hereinbefore, such as obesity, metabolic syndrome, (e.g. type 2) diabetes mellitus, etc (vide infra).

Type 2 diabetes mellitus is a disorder that is characterized by a decreased response of peripheral tissues to insulin (insulin resistance) and beta-cell dysfunction that is manifested as inadequate insulin secretion in the face of insulin resistance and hyperglycemia (see e.g. Robbins and Cotran, Pathologic Basis of Disease, 8^{th} edition, Saunders Elsevier). Symptoms of type 2 diabetes mellitus include chronic fatigue, excessive urine production, excessive thirst and increased fluid intake. The current World Health Organisation diagnostic criteria for diabetes are (a) a fasting plasma glucose level of at least 7.0 mmol/L or (b) a plasma glucose level of at least 11.1 mmol/L in an oral glucose tolerance test (OGTT). By “reducing the development of type 2 diabetes mellitus”, we include prevention of the onset of type 2 diabetes mellitus in addition to treatment of SLGI to prevent development (e.g. worsening) of a pre-existing condition.

We have found that pemirolast does not concomitantly reduce plasma tryptase levels in the subjects with CRP above 0.9 mg/L, and also that there is no correlation between plasma levels of CRP and mast cell tryptase levels in the subjects.

Thus, it is preferred that the uses and methods described herein are in, or of, non-allergic patients. By “non-allergic”, we mean that the patient does not exhibit outward signs (at the time of receiving a treatment according to the invention) of an atopic disorder of the immune system. In this respect, such a patient may show no signs of hypersensitivity to allergens, characterised by a immunological response which includes activation of mast cells and/or basophils via IgE. Determination of whether a patient is non-allergic may be carried out routinely by for example testing (e.g. the skin) for responses to known allergens or analyzing the blood for the presence and levels of allergen-specific IgE.

It is further preferred that the uses and methods described herein are in, or of, non-asthmatic patients. By “non-asthmatic”, we mean that the patient does not exhibit outward signs (at the time of receiving a treatment according to the invention) of predisposition to chronic inflammation of the lungs in which the bronchi are reversibly narrowed by way of constriction of smooth muscle cells therein, airway inflammation and difficulties in breathing. Asthma may be allergic or non-allergic.

Preferred uses and methods of treatment according to the invention include those in which the patient has hypertension or, more preferably, is a smoker or is an ex-smoker, the subject has diabetes mellitus and/or metabolic syndrome, or has a body mass index above 25.

Pharmaceutically-acceptable salts of pemirolast that may be mentioned include acid addition salts and base addition salts. Such salts may be formed by conventional means, for example by reaction of a free acid or a free base form of an active ingredient with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of an active ingredient in the form of a salt with another counter-ion, for example using a suitable ion exchange resin.

Preferred salts of pemirolast include alkaline earth, and more particularly alkali, metal salts, such as calcium, magnesium, preferably sodium and, particularly, potassium salts (e.g. pemirolast potassium).

In the uses and methods described herein, pemirolast and salts thereof are preferably administered locally or systemically, for example orally, intravenously or intraarterially (including by intravascular or other perivascular devices/dosage forms (e.g. stents)), intramuscularly, cutaneously, subcutaneously, transmucosally (e.g. sublingually or buccally), rectally, transdermally, nasally, pulmonarily (e.g. tracheally or bronchially), topically, or by any other parenteral route, in the form of a pharmaceutical preparation comprising the compound in a pharmaceutically acceptable dosage form. Preferred modes of delivery include oral (particularly), intravenous, cutaneous or subcutaneous, nasal, intramuscular, or intraperitoneal delivery.

Pemirolast and salts thereof will generally be administered in the form of one or more pharmaceutical formulations in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Such pharmaceutically acceptable carriers may also impart an immediate, or a modified, release of a compound of the invention.

Suitable pharmaceutical formulations may be commercially available or otherwise are described in the literature, for example, Remington The Science and Practice of Pharmacy, 19th ed., Mack Printing Company, Easton, Pa. (1995) and Martindale—The Complete Drug Reference (35^{th} Edition) and the documents referred to therein, the relevant disclosures in all of which documents are hereby incorporated by reference. Otherwise, the preparation of suitable formulations may be achieved non-inventively by the skilled person using routine techniques.

The amount of pemirolast or salt thereof in the formulation will depend on the severity of the condition, and on the patient, to be treated, as well as the compound(s) which is/are employed, but may be determined non-inventively by the skilled person.

Depending on the disorder, and the patient, to be treated, as well as the route of administration, pemirolast or salt thereof may be administered at varying therapeutically effective doses to a patient in need thereof.

However, the dose administered to a mammal, particularly a human, in the context of the present invention should be sufficient to effect a therapeutic response in the mammal over a reasonable timeframe (as described hereinbefore). One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the age, condition, body weight, sex and response of the patient to be treated, and the stage/severity of the disease, as well as genetic differences between patients.

Administration of pemirolast or salt thereof may be continuous or intermittent (e.g. by bolus injection). The dosage may also be determined by the timing and frequency of administration.

Suitable doses include those referred to in the medical literature, such as Martindale—The Complete Drug Reference (35^{th} Edition) and the documents referred to therein, the relevant disclosures in all of which documents are hereby incorporated by reference. Suitable doses of pemirolast or salt thereof (calculated as the free acid) are therefore in the range of about 0.01 mg/kg of body weight to about 1,000 mg/kg of body weight. More preferred ranges are about 0.1 mg/kg to about 20 mg/kg on a daily basis, when given orally.

However, suitable doses of pemirolast are known to those skilled in the art. For example, peroral doses (calculated as the free acid) may be in the range of about 0.1 mg to about 1.2 g, such as about 0.5 mg to about 900 mg, per day. For example suitable lower limits of daily dose ranges are about 1 mg, such as about 2 mg, for example about 5 mg, such as about 10 mg, and more preferably about 20 mg; and suitable upper limits of daily dose ranges are about 200 mg, for example about 100 mg, such as about 80 mg. Daily peroral doses may thus be between about 2 mg and about 100 mg (e.g. about 50 mg), such as about 5 mg and about 60 mg (e.g. about 40 mg), and preferably about 10 mg and about 50 mg (e.g. about 30 mg). Suitable individual doses may be about 40 mg, or, more preferably, about 30 mg (such as about 25 mg).

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

In the uses and methods described herein, pemirolast and pharmaceutically acceptable salts thereof may also be combined with one or more active ingredients that are useful in the treatment of cardiovascular morbidity and mortality, and/or of type 2 diabetes mellitus. Such patients may thus also (and/or already) be receiving therapy based upon administration of one or more of such active ingredients, by which we mean receiving a prescribed dose of one or more of those active ingredients mentioned herein, prior to, in addition to, and/or following, treatment with pemirolast or salt thereof.

Such active ingredients include thromboxane A2 antagonists, P2Y₁₂ antagonists, PPAR_{γ} agonists, compounds that inhibit the formation and/or action of angiotensin II, other platelet aggregation inhibiting drugs, anti-diabetic drugs, lipid-lowering drugs and, more preferably, statins.

The term “thromboxane A2 antagonist” includes any compound that is capable of inhibiting, to an experimentally-determinable degree in in vitro and/or in vivo tests, the effects of thromboxane A2 by one or more of (i) blocking the thromboxane TP receptor, (ii) inhibiting the enzyme thromboxane synthase, or (iii) inhibiting (e.g. selectively) platelet cyclooxygenase-1, thereby inhibiting e.g. platelet aggregation.

Preferred thromboxane A2 antagonists include seratrodast, more preferably egualen, particularly ozagrel, more particularly, picotamide and terutroban, especially aspirin/acetylsalicylic acid and more especially ramatroban.

The term “P2Y₁₂ antagonist” includes any compound that is capable of inhibiting (e.g. selectively), to an experimentally-determinable degree in in vitro and/or in vivo tests, the binding of ADP to the platelet receptor P2Y₁₂, thereby inhibiting platelet aggregation.

Preferred P2Y₁₂ antagonists include prasugrel, ticagrelor and, particularly, clopidogrel.

The term “PPAR_{γ}” agonist includes any compound that is capable of binding to, and/or influencing the function of, the peroxisome proliferator-activated gamma receptor to an experimentally-determinable degree in in vitro and/or in vivo tests.

Preferred PPAR_{γ} agonists therefore include the compounds collectively known together as thiazolidinediones, including rivoglitazone, naveglitazar, balaglitazone or, more preferably, rosiglitazone and, especially, pioglitazone. Other PPAR_{γ} agonists that may be mentioned include chiglitazar, etalocib, farglitazar, lobeglitazone, netoglitazone, sodelglitazar, as well as those defined in the literature by way of following developmental drug codes: THR-0921 (Theracos Inc.) or, more preferably, AVE-0847 and AVE-0897 (both Sanofi-Aventis), CLX-0921 (Calyx Therapeutics), CS-7017 (Daiichi Sankyo Co Ltd), DRF-11605 (Dr Reddy's Laboratories Ltd), GFT-505 (Genfit SA), GSK-376501 (GlaxoSmithKline plc), INT-131 (Amgen Inc; InteKrin Therapeutics), (LBM-642; cevoglitazar; Novartis AG), ONO-5129 (Ono Pharmaceutical Co Ltd), (PDC-204; indeglitazar; Plexxikon Inc) and SDX-101.

The term “compound that inhibits the formation and/or action of angiotensin II” includes any compound that is capable of inhibiting (e.g. selectively), to an experimentally-determinable degree in in vitro and/or in vivo tests, the formation and/or action of angiotensin II and will be understood to include angiotensin converting enzyme (ACE) inhibitors, angiotensin receptor blockers (ARBs) and renin inhibitors.

The term “angiotensin converting enzyme (ACE) inhibitor” includes any compound that is capable of inhibiting (e.g. selectively), to an experimentally-determinable degree in in vitro and/or in vivo tests, the conversion of angiotensin I to angiotensin II.

ACE inhibitors that may be mentioned include alacepril, benazepril, captopril, ceronapril, cilazapril, delapril, enalapril, fosinopril, gemopatrilat, glycopril, idrapril, ilepatril, imidapril, libenzapril, lisinopril, microginin-FR1, mixanpril, moexipril, moexiprilat, moveltipril, omapatrilat, Prentyl, perindopril, quinapril, ramipril, sampatrilat, spirapril, Synecor, temocapril, trandolapril, utibapril, zofenopril and zabiciprilat. More preferred ACE inhibitors include benazepril, cilazapril, ilepatril, imidapril, moexipril, spirapril, temocapril and zofenopril, more preferably fosinopril and trandolapril, more particularly enalapril, lisinopril and quinapril, and especially captopril, perindopril and ramipril.

The term “angiotensin receptor blocker (ARB)” will be understood by the skilled person to be largely synonymous with the term “angiotensin II AT1 receptor antagonist”, and thus includes any substance that is capable of blocking the activation (e.g. selectively), to an experimentally-determinable degree in in vitro and/or in vivo tests, of the angiotensin II AT1 receptor.

ARBs that may be mentioned include azilsartan, azilsartan medoxomil, candesartan, candesartan cilexetil, the angiokine Dival, elisartan, elisartan potassium, eprosartan, embusartan, fimasartan, fonsartan, irbesartan, losartan, milfasartan, olmesartan, pomisartan, pratosartan, ripisartan, saprisartan, saralasin, tasosartan, telmisartan, valsartan and zolasartan. More preferred ARBs include azilsartan, eprosartan, fimasartan and pratosartan, more preferably telmisartan, more particularly irbesartan and olmesartan, and especially candesartan, losartan and valsartan.

The term “renin inhibitor” will be understood by the skilled person to includes any substance that is capable of blocking the function (e.g. selectively), to an experimentally-determinable degree in in vitro and/or in vivo tests, of renin in the renin-angiotensin system.

Renin inhibitors that may be mentioned include cyclothiazomycin, aliskiren, ciprokiren, ditekiren, enalkiren, remikiren, terlakiren and zankiren. Preferred renin inhibitors include aliskiren.

Compounds that inhibit the formation and/or action of angiotensin II also include those defined in the literature by way of the following developmental drug codes: 100240, 606A, A-65317, A-68064, A-74273, A-81282, A-81988, A-82186, AB-47, BIBR-363, BIBS-222, BIBS-39, BILA-2157BS, BL-2040, BMS-180560, BMS-181688, BMS-182657, BMS-183920, BMS-184698, BRL-36378, CGP-38560, CGP-38560a, CGP-42112-A, CGP-42112, CGP-421132-B, CGP-48369, CGP-49870, CGP-55128A, CGP-56346A, CGS-26670, CGS-26582, CGS-27025, CGS-28106, CGS-30440, CHF-1521, CI-996, CL-329167, CL-331049, CL-332877, CP-191166, CP-71362, CV-11194, CV-11974, DMP-581, DMP-811, DU-1777, DuP-167, DuP-532, E-4030, E-4177, EC-33, EK-112, EMD-56133, EMD-58265, EMD-66684, ER-32897, ER-32935, ER-32945, ES-1005, ES-305, ES-8891, EXP-408, EXP-597, EXP-6803, EXP-7711, EXP-929, EXP-970, FPL-66564, GA-0050, GA-0056, GA-0113, FK-739, FK-906, GR-137977, GR-70982, GW-660511, Hoe-720, ICI-219623, ICI-D-6888, ICI-D-8731, JT-2724, KR-30988, KRH-594, KRI-1314, KT3-866, KW-3433, L-158809, L-158978, L-159093, L-159689, L-159874, L-159894, L-159913, L-161177, L-161290, L-161816, L-162223, L-162234, L-162313, L-162389, L-162393, L-162441, L-162537, L-162620, L-163007, L-163017, L-163579, L-163958, L-363564, L-746072, LCY-018, LR-B-057, LY-285434, LY-301875, LY-315996, MDL-102353, MDL-27088, MDL-27467A, ME-3221, MK-8141, MK-996, PD-123177, PD-123319, PD-132002, PD-134672, PS-433540, RB-106, RS-66252, RU-64276, RU-65868, RWJ-38970, RWJ-46458, RWJ-47639, RXP-407, S-2864, S-5590, SB-203220, SC-50560, SC-51316, SC-51895, SC-52458, SC-54629, SC-565254, Sch-47896, Sch-54470, SK-1080, SKF-107328, SL-910102, SQ-30774, SQ-31844, SQ-33800, SR-43845, TA-606, TH-142177, U-97018, UK-63831, UK-77568, UK-79942, UP-275-22, WAY-121604, WAY-126227, VNP-489, XH-148, XR-510, YM-21095, YM-26365, YM-31472, YM-358 and ZD-7155.

Other platelet aggregation inhibiting drugs that may be mentioned include nitric oxide-donating derivatives of aspirin/acetylsalicylic acid (e.g. NCX-4016, NicOx S.A.) or, more preferably, anagrelide, argatroban, beraprost, cangrelor, cilostazol, dipyridamole, limaprost, parogrelil, procainamide, sarpogrelate (e.g. sarpogrelate hydrochloride), ticlopidine, tirofiban and triflusal, as well as those defined in the literature by way of following developmental drug codes: DA-697b (see international patent application WO 2007/032498; Daiichi Seiyaku Co Ltd), DG-041 (deCODE Genetics Inc), K-134 (CAS RN 189362-06-9), PL-2200 (CAS RN 50-78-2), PRT-60128 (Portola Pharmaceuticals Inc), SH-529 (an iloprost/beta-cyclodextrin clathrate; Bayer Schering Pharma AG) and YY-280 (a combination therapy of ticlopidine and EGb-761 (tanamin; a Ginkgo biloba extract; Yuyu Inc.)).

Lipid-lowering drugs include resins (such as cholestyramine, colesevelam, colestipol, or any other drug that acts by binding bile acids, so causing the liver to produce more of the latter and using up cholesterol in the process); the B-vitamin niacin, fibrates (such as bezafibrate, ciprofibrate, clofibrate, gemfibrozil and fenofibrate), or any other drug that is capable of lowering triglyceride levels, lowering LDL levels and/or increasing HDL levels; and ezetimibe, or any other drug which acts by inhibiting the absorption of cholesterol from the intestine.

The term “statin” includes any inhibitor of HMG-CoA reductase and includes fluvastatin, simvastatin, lovastatin, rosuvastatin, pitavastatin, glenvastatin, cerivastatin, pravastatin, mevastatin, bervastatin, dalvastatin and atorvastatin.

Other statins that may be mentioned include Acitemate, benfluorex, Clestin, colestolone, dihydromevinolin, meglutol, rawsonol, as well as the compounds with the following code names: ATI-16000, BAY-10-2987, BAY-x-2678, BB-476, B10-002, B10-003, B10-2, BMS-180431, CP-83101, DMP-565, FR-901512, GR-95030, HBS-107, KS-01-019, L-659699, L-669262, NR-300, P-882222, PTX-023595, RP 61969, S-2468, SC-32561, sc-45355, SDZ-265859, SQ-33600, U-20685, and NO-enhancing/releasing statins, such as NCX-6550 (nitropravastatin) and NCX-6560 (nitroatorvastatin).

More preferred statins include pitavastatin (e.g. Livalo®, Pitava®), fluvastatin (e.g. Lescol®), simvastatin (e.g. Zocor®, Lipex®), lovastatin (e.g. Mevacor®, Altocor®), rosuvastatin (e.g. Crestor®), pravastatin (e.g. Pravachol®, Selektine®, Lipostat®) and atorvastatin (e.g. Lipitor®, Torvast®). Particularly preferred statins include simvastatin, more particularly atorvastatin and, especially, rosuvastatin.

Pharmaceutically-acceptable salts of other active ingredients useful in the treatment of cardiovascular morbidity and mortality that may be mentioned include acid addition salts and base addition salts. Such salts may be formed by conventional means, for example as described hereinbefore for pemirolast.

Salts of picotamide that may be mentioned include hydrochloride, bisulfate, maleate and tosylate salts. Salts of ozagrel, terutroban, egualen and aspirin that may be mentioned include alkali metal salts, such as lithium, sodium and potassium salts. Preferred salts of ozagrel and egualen include sodium salts.

Preferred salts of clopidogrel include bisulfate salts, but other salts that may be mentioned, as well as salts of ticagrelor that may be mentioned, include hydrochloride, bisulfate, maleate and tosylate salts. Preferred salts of prasugrel that may be mentioned include hydrochloride salts, but other salts that may be mentioned include bisulfate, maleate and tosylate salts.

Preferred salts of pioglitazone that may be mentioned include hydrochloride salts, but other salts that may be mentioned include bisulfate, maleate and tosylate salts. Preferred salts of rosiglitazone that may be mentioned include maleate salts, but other salts that may be mentioned include hydrochloride, bisulfate and tosylate salts. Salts of rivoglitazone that may be mentioned include hydrochloride, bisulfate, maleate and tosylate salts. Preferred salts of naveglitazar include sodium salts, but other salts that may be mentioned include lithium and potassium salts. Preferred salts of balaglitazone that may be mentioned include sodium, potassium and calcium salts.

Preferred salts of compounds that inhibit the formation and/or action of angiotensin II include, for example, hydrochloride, bisulfate, maleate, mesylate, tosylate, alkaline earth metal salts, such as calcium and magnesium, or alkali metal salts, such as sodium and potassium salts. Such salts may be prepared using routine techniques for compounds including perindopril, enalapril, lisinopril, quinapril, irbesartan, olmesartan, trandolapril, telmisartan, benazepril, cilazapril, moexipril, spirapril, eprosartan and fimasartan. Hydrochloride, bisulfate, maleate, mesylate and tosylate salts are preferred for compounds such as ramipril and aliskiren. Alkaline earth, and more particularly alkali, metal salts are preferred for compounds such as candesartan, valsartan, captopril, losartan and, particularly, fosinopril, preferred salts of which include calcium, magnesium, potassium and, particularly, sodium salts. Preferred salts of benazepril and moexipril that may be mentioned include hydrochloride salts, but other salts that may be mentioned include bisulfate, maleate, mesylate and tosylate salts. Preferred salts of eprosarten that may be mentioned include mesylate salts, but other salts that may be mentioned include hydrochloride, bisulfate, maleate and tosylate salts.

Preferred salts of statins include sodium, potassium and calcium salts, such as pitavastatin calcium, fluvastatin sodium, pravastatin sodium, rosuvastatin calcium and atorvastatin calcium.

Suitable doses of other active ingredients include those that are useful in the treatment of cardiovascular disorders (or diabetic disorders, as appropriate), and particularly cardiovascular morbidity and mortality and/or type 2 diabetes mellitus, are known to those skilled in the art and include those listed for the drugs in question to in the medical literature, such as Martindale—The Complete Drug Reference (35^{th} Edition) and the documents referred to therein, the relevant disclosures in all of which documents are hereby incorporated by reference.

Wherever the word “about” is employed herein, for example in the context of amounts (e.g. plasma CRP levels and doses of active ingredients), it will be appreciated that such variables are approximate and as such may vary by ±10%, for example ±5% and preferably ±2% (e.g. ±1%) from the numbers specified herein.

The uses/methods described herein may have the advantage that, in the treatment of SLGI, they may be more convenient for the physician and/or patient than, be more efficacious than, be less toxic than, have a broader range of activity than, be more potent than, produce fewer side effects than, or that it may have other useful pharmacological properties over, similar methods (treatments) known in the prior art for use in such therapy.

The invention is illustrated, but in no way limited, by the following example, in which:

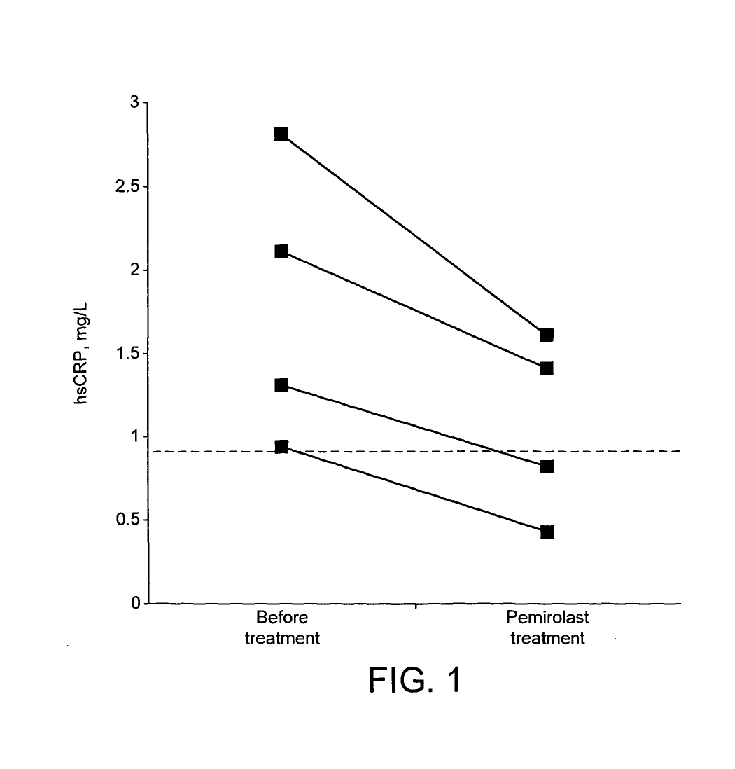

FIG. 1 illustrates plasma CRP levels in four healthy volunteers with SLGI before and after treatment with pemirolast potassium for five days. The dotted line indicates the cut-point at 0.9 mg/L plasma CRP.

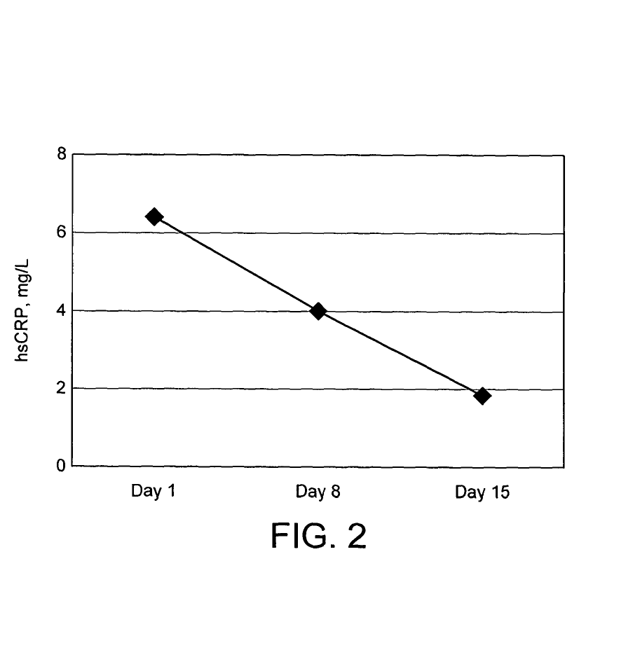

FIG. 2 illustrates plasma CRP levels in a patient with a cardiovascular disease during treatment with pemirolast potassium over fourteen days.

### EXAMPLE 1

### Reduction of SLGI by Peroral Pemirolast Treatment

The study was approved by the Swedish Medical Products Agency and performed by Berzelius Clinical Research Centre AB in LinkOping, Sweden.

The objectives of this study were to determine the pharmacokinetics, safety and tolerability of orally administered pemirolast (10, 30 or 50 mg b.i.d. as described below). Briefly, the results showed that pemirolast was well tolerated, the absorption was relatively rapid, and AUC and Cmax increased in a dose-proportional manner. From a safety perspective, there were no clinically important findings in laboratory values, vital signs or ECG. However, it was surprisingly found that pemirolast reduced plasma CRP levels in patients with hsCRP levels >0.9 mg/L (i.e. those with SLGI, as described above).

Plasma CRP was determined with a high sensitivity CRP assay (based on Near Infrared Particle Immunoassay rate methodology) on a UniCel DxC 800 instrument from Beckman Coulter (analysis performed at the Department of Clinical Chemistry, Karolinska University Laboratory, Stockholm, Sweden). The degree of mast cell activity was determined by measuring plasma tryptase with the ImmunoCAP Tryptase Fluoro-Immuno-Enzymatic Assay on a ImmunoCAP 250 instrument from Phadia (analysis performed at Clinical Immunology and Transfusion Medicine, Karolinska University Laboratory, Stockholm, Sweden).

Blood sampling for the plasma analyses were performed immediately before the first pemirolast dose (CRP and tryptase) and two (tryptase) or four (CRP) hours after the last dose (the plasma levels of pemirolast remained essentially stable during the sampling period after the last dose).

Seventeen non-allergic healthy volunteers (all males, age 18-45 years, mean age 25 years) were treated per orally with 10 mg (n=6), 30 mg (n=5) or 50 mg (n=6) pemirolast potassium (10 mg Ulgixal tablets purchased from Taiyo Pharmaceutical Industry Co., Ltd, Japan). Each subject received the first pemirolast dose in the morning of day 1. During days 2-4, each subject received one dose in the morning and one dose in the evening with a time interval of 12 hours between the daily doses. The last (8^{th}) dose of pemirolast was administered in the morning of day 5. Other than the occasional use of nasal decongestants or paracetamol, the subjects were instructed not to use other drugs, alcohol or nicotine during the study.

Four of the seventeen subjects had plasma CRP levels above 0.9 mg/L (i.e., as discussed herein, indicative of SLGI at a level above which the risk for cardiovascular events has been shown to be increased, see Ridker et al, N. Engl. J. Med., 352, 20 (2005)) before pemirolast treatment.

Pemirolast (10 mg (one subject), 30 mg (one subject), or 50 mg (two subjects) administered during day 1-5 as described above) markedly reduced the CRP levels in these four subjects (see FIG. 1). The mean CRP level in these four subjects was significantly reduced from 1.8 mg/L before treatment to 1.1 mg/L at the end of the pemirolast treatment (p<0.05). In the remaining subjects with baseline levels of CRP below 0.9 mg/L, the mean CRP level was 0.32 mg/L before treatment and 0.38 mg/L at the end of the pemirolast treatment (n=13).

To determine whether plasma tryptase levels (reflecting the degree of mast cell activity) correlated with CRP levels, tryptase levels were also analysed before and after pemirolast treatment. The levels of tryptase were between 1.8 and 14 μg/L and there was no trend for positive or negative correlation between plasma CRP and tryptase (correlation coefficient 0.001). In the four subjects that had SLGI with plasma CRP >0.9 mg/L, and in which pemirolast reduced the SLGI, pemirolast treatment did not reduce the tryptase levels, i.e. the mean plasma tryptase level was 4.8 μg/L both before and after pemirolast treatment. This suggests that the inhibitory effect of pemirolast on SLGI was unrelated to mast cell inhibition. (These two observations together provide further evidence (in addition to the prior art disclosures discussed hereinbefore) that mast cells are not involved in the origin of SLGI.)

### EXAMPLE 2

### Reduction of SLGI by Peroral Pemirolast Treatment in a Patient with Cardiovascular Disease

The objective of this study was to assess the effect of pemirolast on the plasma C-reactive protein (CRP) level in a patient with cardiovascular disease, and more specifically a patient with coronary artery disease (CAD). The study was approved by the Swedish Medical Products Agency, and performed by Berzelius Clinical Research Centre AB in Linköping, Sweden (the clinic).

Plasma CRP was determined with the high sensitivity CRP (hsCRP) assay described in Example 1 above.

The CAD patient (see below) was treated with 30 mg pemirolast potassium (10 mg Ulgixal tablets purchased from Taiyo Pharmaceutical Industry Co., Ltd, Japan) b.i.d. for two weeks. The first dose (3×10 mg) was administered in the morning of Day 1 at the clinic, the second dose in the evening of Day 1 at home, and then 3×10 mg b.i.d. at home during Days 2 to 14. The last dose was administered in the evening of Day 14 at home. Blood sampling for plasma CRP analyses was performed immediately before the first pemirolast dose on Day 1, in the morning of Day 8 (after the morning dose of pemirolast that day) and in the morning of Day 15.

The CAD patient was a 63 year old Caucasian male with a body height of 188 cm and a body weight of 103 kg. He was on treatment with enalapril 20 mg QD (once daily) for hypertension since 2006. After a myocardial infarction in 2009, he was started on treatment with simvastatin 40 mg QD, acetylsalicylic acid 75 mg QD, and metoprolol 100 mg QD (all three drugs started in June 2009). Since January 2010, the patient was also treated with felodipin 5 mg QD. During the two week treatment with pemirolast, the patient continued to take these medications. Upon entering the study, the patient had a normal physical examination and ECG.

On Day 1 (before the first dose of pemirolast), the patient had a CRP level of 6.4 mg/L. On Day 8 of pemirolast treatment, the CRP level was reduced to 4.0 mg/L, and in the morning of Day 15, the CRP level was down to 1.8 mg/L (see FIG. 2).

The patient did not report any adverse events during the study.

1. (canceled)

2. (canceled)

3. A method of treatment of systemic low-grade inflammation, which method comprises the administration of pemirolast, or a pharmaceutically acceptable salt thereof, to a patient in need of such treatment.

4. A method as claimed in claim 1, wherein the systemic low grade inflammation is characterised by plasma C-reactive protein levels above about 0.9 mg/L.

5. A method of reducing plasma C-reactive protein levels in a patient, which method comprises the administration of pemirolast, or a pharmaceutically acceptable salt thereof, to that patient.

6. A method of reducing the risk of cardiovascular morbidity and/or mortality in a patient, which method comprises:
(a) measuring a plasma C-reactive protein level in that patient;
(b) determining whether that plasma C-reactive protein level is above about 0.9 mg/L; and
(c) if so, administering pemirolast, or a pharmaceutically acceptable salt thereof, to that patient for a time and at an appropriate dosage to reduce the plasma C-reactive protein level.

7. A method of reducing the development of type 2 diabetes mellitus in a patient, which method comprises:
(a) measuring a plasma C-reactive protein level in that patient;
(b) determining whether that plasma C-reactive protein level is above about 0.9 mg/L; and
(c) if so, administering pemirolast, or a pharmaceutically acceptable salt thereof, to that patient for a time and at an appropriate dosage to reduce the plasma C-reactive protein level.

8. A method as claimed in claim 3 wherein the patient is non-allergic and/or non-asthmatic.

9. A method as claimed in claim 3 wherein the patient has hypertension, is a smoker or an ex-smoker, has diabetes mellitus, has metabolic syndrome, and/or has a body mass index above 25.

10. A method as claimed in claim 3, wherein the patient is also receiving therapy which comprises administration of an active ingredient selected from a thromboxane A2 antagonist, a P2Y₁₂ antagonist, a PPAR_{γ} agonist, a compound that inhibits the formation and/or action of angiotensin II, a platelet aggregation inhibiting drug and a statin.

11. A method as claimed in claim 10, wherein the active ingredient is a statin.

12. A method as claimed in claim 11, wherein the active ingredient is atorvastatin or rosuvastatin.

13. A method as claimed in claim 10, wherein the active ingredient is aspirin/acetylsalicylic acid, egualen, ozagrel, picotamide, terutroban, seratrodast, ramatroban, prasugrel, ticagrelor, clopidogrel, rivoglitazone, naveglitazar, balaglitazone, rosiglitazone, pioglitazone, captopril, perindopril, ramipril, candesartan, losartan, valsartan or aliskiren.
